(19) United States
(12) Patent Application Publication
(10) Pub. No.: US 2002/0010445 A1
(43) Pub. Date: Jan. 24, 2002

(54) Ostomy bag cover

(76) Inventor: Doris M. Gunn, San Antonio, TX (US)

Correspondence Address: Daniel D. Chapman, JACKSON WALKER, LLP, Suite 2100, 112 E. Pecan, San Antonio, TX, 78205, US

(21) Appl. No.: 09/909,435

(22) Filed: Jul. 19, 2001

Related U.S. Application Data
(60) Provisional application No. 60/219,382, filed on Jul. 19, 2000.

(51) Int. Cl. A61F 5/44

(52) U.S. Cl. 604/345

(57) ABSTRACT

Applicant's invention provides an ostomy bag cover for containing within an ostomy bag. The cover has a distal panel and an proximal panel with an optional belt attachment. The two panels are of similar size and shape and are placed together and joined, as by sewing for example, along their perimeters. However, the two (2) joined panels, forming a cover, have an opening at top for accepting the ostomy bag and an opening along the bottom, for emptying the ostomy bag.

This application is based on and maintains priority from provisional patent application Ser. No. 60/219,382, filed under 35 C.F.R. 1.53(b)(2), on Jul. 19, 2000.

### BACKGROUND OF THE INVENTION

Field of The Invention

An ostomy bag cover, more specifically, an ostomy bag cover for providing a functional but aesthetically pleasing protective cover to an ostomy bag.

Background Information

After undergoing an ostomy surgery, wherein an opening is created to provide removal of waste from within the body, an ostomy bag may be required to be worn for collection of these secretions. These bags are typically transparent or semi-transparent. Usually, the bags have a flanged opening in the upper section thereof, and a drain opening in the lower portion. The upper portion of the bag receives the waste from the body and the lower portion can be opened to release the waste.

### SUMMARY OF THE INVENTION

Applicant's invention provides an ostomy bag cover for cooperation with an ostomy bag, the cover comprising a distal panel and a proximal panel with an optional belt attachment. The proximal panel, which is shaped and sized similar to the distal panel, rests against the wearer and provides a separation layer between the bag and the wearer. The proximal panel, providing the separation layer, may be constructed of a double-layer of fabric or other suitable material, including cotton material for comfort. The distal panel may be constructed of satin, cotton, terry cloth or other typically opaque material, or a combination of these materials to provide a pleasing external view of the cover. Additionally, all materials or fabrics used in the construction of the cover are preferably made of washable materials.

The proximal panel includes an opening therein for encircling the ostomy bag when the bag is attached to the body. The opening is comprised of two arms with a releaseable means of attachment, including but not limited to velcro or the like. These arms surround the flanged opening of the ostomy bag and may provide for ties for direct attachment to ostomy bags which are designed with eyelets. These eyelets allow for the cover to be attached to the bag by a means of horizontally opposed ties affixed to the arms. These ties provide means for securing the cover to the bag to prevent accidental removal of the cover from the bag.

Some bags may omit the eyelets. In this case, the cover provides a top closure which adjoins the proximal and distal panels at the apex of the curve. This top attachment prevents the removal of the cover from the bag.

The bottom opening of the cover is created by the joining of the two panel along an outer edge perimeter seam. This seam creates an opening at the bottom of the cover for the removal of waste from the bag.

Furthermore, the cover may include a belt, typically worn around the waist and attached to the top of the cover. This allows the cover to be held in position on the bag while being worn. The belt may be removed from the rest of the cover by means of a releaseable attachment.

The present invention may include a decorative design on any portion thereof. This design may include lace, ruffles or the like, which may be affixed to the perimeter of the panels. Additionally, design elements may be incorporated on the face of the proximal or distal panels.

### DESCRIPTION OF THE DRAWINGS

FIG. 1 is a front elevation view of the proximal side of the ostomy bag cover;

FIG. 1A is a front elevation view of the proximal side of a typical ostomy bag illustrated here with eyelets;

FIG. 1B is a cross sectional view of a typical ostomy bag with Applicant's cover attached thereto;

FIG. 2 is a left side elevational view of the cover;

FIG. 3 is an elevation view of the proximal panel of the cover; and

FIG. 4 is a perspective view of the cover illustrating the attachment of the belt to the proximal panel of the cover.

### DESCRIPTION OF THE PREFERRED EMBODIMENT

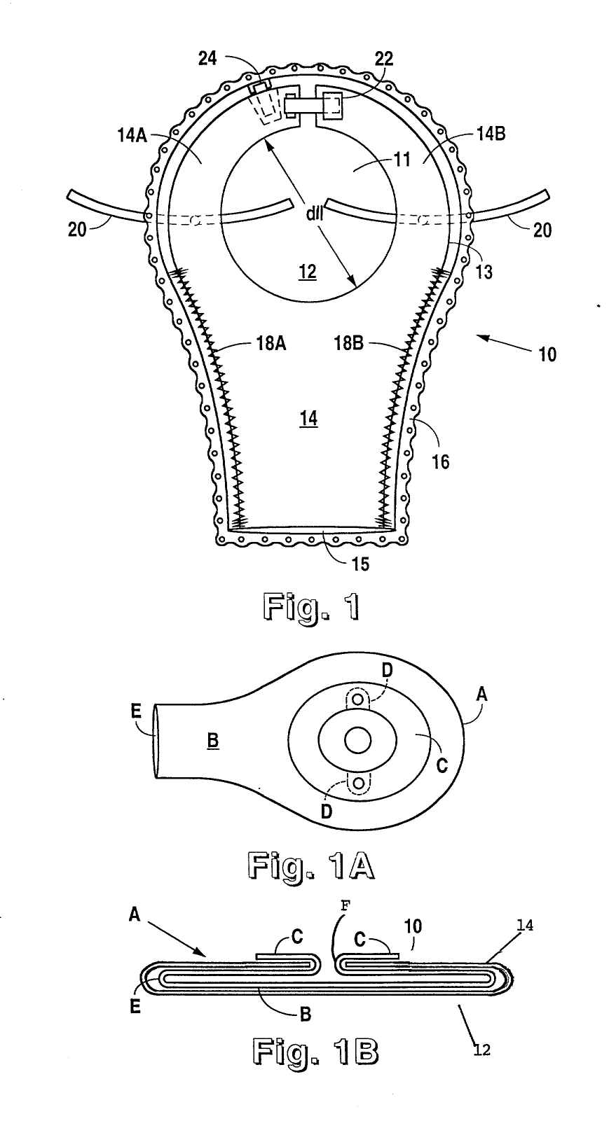

With reference to the figures, FIG. 1A and 1B show a typical ostomy bag A (FIG. 1A showing optional eyelets D). The ostomy bag (A) has a body (B) with lower opening E, sealing member C for adhesively and releasably sealing against the skin of the wearer and a neck F between the body B and sealing member C.

FIGS. 1,2,3, and 4 refer to an ostomy bag cover 10 for use with the ostomy bag (A) by ostomy patients. The cover consists of a distal panel 12 and a proximal panel 14 and an upper opening 13 between the two panels for insertion of a ostomy bag between the two panels (See also FIG. 1B). Additionally, the cover depicts optional decorative border lace 16 and decorative panel design lace 16a used in conjunction with the cover.

Referring to FIG. 1, this view depicts the proximal side of the cover (the side of the cover that will rest against the skin) and the opening 13 created by the arms 14A and 14B, which arms form the upper portion of the proximal panel 14.

The arms 14A and 14B are releasably attached to one another at their removed ends by arms attachment means 22 (such as Velcro®) for engaging and disengaging cover with the ostomy bag A. Attachment means 22 allows for the arms 14A and 14B to detach and reattach to form the upper opening 11, typically circular, allowing for encircling of the neck F of an ostomy bag (See FIG. 1B). Horizontally centered on the upper opening 11 on each arm 14 are, optionally, ties 20 which may be utilized to attach the cover to an ostomy bag, providing that such bag allows for such attachment, such as eyelets D adjacent the flange portion (See FIG. 1A).

About midway between the top and the bottom of the proximal panel 14, seams 18A and 18B are located along (typically adjacent the lower arc of the upper opening 11) the right and left side of the cover providing for the attachment of the proximal panel 14 to the distal panel 12. These seams extend from about the widest portion of the panels 12,14 to the bottom edge of both panels 12,14, typically without sealing the bottom edge, thereby creating a bottom opening 15. The upper sections of the panels 12,14 are connected to one another by means of a top connection 24 (such as Velcro®). This connection is also capable of being released and reattached for access to the bag and for joining the bag to the cover.

Distal panel 12 faces away from the wearer and may be viewed by others when the cover is in use on the wearer. Thus, FIG. 3 illustrates a possible decorative lace 16 attached to the perimeter of the distal panel 12. Further depicted is a decorative design lace 16a which may be attached across the face of distal panel 12. Indeed, Applicant provides for a variety of material and decorating for the face of the distal panel, those illustrated being only examples of a wide variety of possible designs.

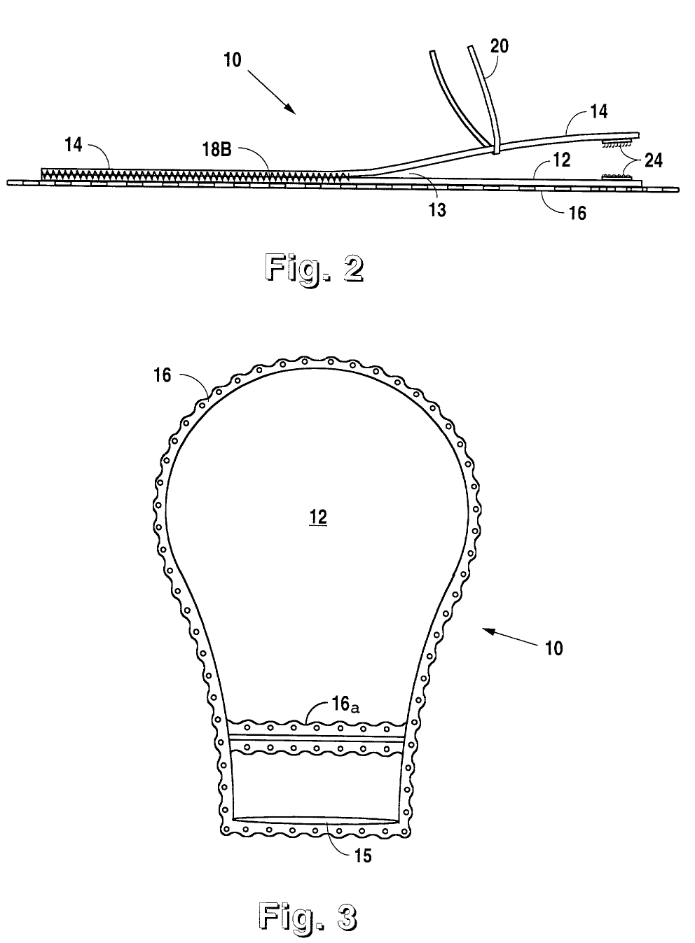

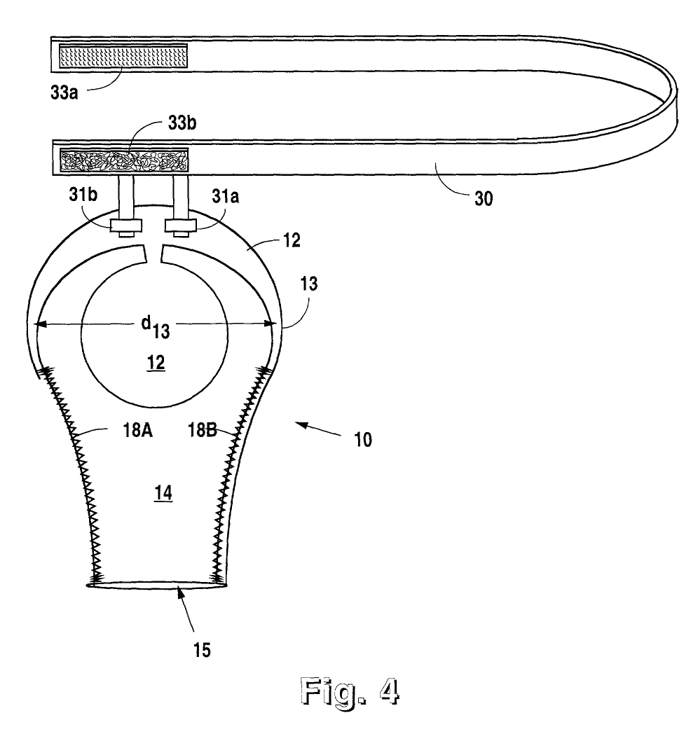

FIG. 4, illustrates the optional attachment of a belt 30, which is typically worn around the waist of the wearer, for support of the cover 10. This belt 30 may be releasably attached to the cover by means of paired attachments 31a/31b, such as Velcro®. Further, this belt adjusts to different patient body sizes by means of long paired strips of velcro 33a/33b placed along the removed ends of belt 30.

The cover is utilized by inserting the ostomy bag into the cover between between the panels by releasing the top attachment 24 holding the apex of the two panels together. Next, the arms attachment 22 which holds the arms 14A and 14B of the proximal panel 14 together is released. This permits the easy easy insertion of a ostomy bag into the cover with the bottom of the bag extending to the bottom opening 15 and the flange of the bag protruding out of the upper opening 11 that is reformed by the attachment of the arm velcro attachments 22.

If the neck portion (F) of the ostomy bag contains eyelets, the cover may be secured to these eyelets by means of the ties 20. Alternatively, if the bag does not contain these eyelets, the bag may be held in place by means of the belt 30 attached to the cover by velcro attachments 31a/31b.

There are three openings in cover 10. The arms 14A and 14B define upper opening 11, of the bag which will encircle snugly the neck of the bag allowing the arms to lay between sealing member C and body B. Thus the diameter of this opening d11 is slightly greater than the diameter of the neck of the ostomy bag.

The second opening 13 is defined by the upper limit of the two seams 18A and 18B and, has a diameter d13 that is slightly greater than the widest part of the bag.

The third opening, bottom opening 15 is slightly wider than the mouth of the body of the colostomy bag. It is provided for flushing the ostomy bag without removing the cover.

Although the invention has been described with reference to specific embodiments, this description is not meant to be construed in a limiting sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

I claim:

1. An ostomy bag cover for an ostomy bag, the bag having a distal panel and a proximal panel, the proximal panel having an opening therein, the opening for receiving waste from the body of an ostomy patient, with the proximal panel for resting adjacent the patient and the distal panel for facing away from the patient, the two panels of varying widths being joined along a perimeter thereof but open at the bottom, the ostomy bag cover comprising:
a distal panel shaped to conform to the shape of the distal panel of the ostomy bag but being dimensioned to be slightly larger than the distal panel of the bag;
a proximal panel for laying flush against the distal proximal panel of the bag, the proximal panel including a pair of arms defining a bag opening, the pair of arms including a means to releasably join the two arms to one another at the removed ends thereof, the proximal panel including means to attach at least one of the two arms releasably to the distal panel, the proximal panel having the same shape and size as the distal panel; and
a first and a second seam joining the two panels and defining at the removed ends of the two panels, a lower opening, the two seams joining the two panels along the outer edges of thereof and defining a top opening capable of receiving the bag therein.

2. The ostomy bag cover according to claim 1, wherein said ostomy bag contains eyelets, and wherein the proximal panel of the bag includes ties for attaching the cover to the bag.

3. The ostomy bag cover according to claim 1, wherein said cover includes a decorative design.

4. The ostomy bag cover according to claim 1, wherein said cover includes a belt for supporting the joined panels to the wearer.

5. The ostomy bag cover according to claim 4 wherein said belt includes means to reasonably attach the belt to the user.

6. The ostomy bag cover according to claim 5 wherein the belt includes means to size the belt to the wearer.

7. The ostomy bag cover according to claim 1 wherein the top opening of said first and said second seams is slightly larger than the widest point of the ostomy bag.

8. The ostomy bag cover according to claim 1 wherein the opening defined by the pair of arms of the proximal panel is greater that the opening of the proximal panel of the ostomy bag but less than the width of the proximal panel of the ostomy bag as measured at the opening.

9. The ostomy bag cover according to claim 1 wherein the distal panel is constructed at least partially, from one of the following: satin, cotton or terry cloth.

10. The ostomy bag cover of claim 3 wherein the decorative design is located on a perimeter of said distal panel.

11. The ostomy bag cover of claim 3 wherein the decorative design is located on a face of said distal panel.
